(19) United States
(12) Patent Application Publication
(10) Pub. No.: US 2020/0355762 A1
(43) Pub. Date: Nov. 12, 2020

(54) MAGNETIC RESONANCE ELASTOGRAPHY APPARATUS WITH A MAGNETIC RESONANCE UNIT AND AN ELASTOGRAPHY UNIT

(71) Applicant: Siemens Healthcare GmbH, Erlangen (DE)

(72) Inventors: Sabrina Kreher, Langensendelbach (DE); Markus Petsch, Adelsdorf (DE)

(73) Assignee: Siemens Healthcare GmbH, Erlangen (DE)

(21) Appl. No.: 16/870,018

(22) Filed: May 8, 2020

(30) Foreign Application Priority Data
May 10, 2019 (EP) 19173755.0

(51) Int. Cl. G01R 33/30; G01R 33/34; A61B 5/00; A61B 5/055

(52) U.S. Cl. CPC G01R 33/30; G01R 33/34007; A61B 5/0555; A61B 5/055; A61B 5/0051

(57) ABSTRACT

Techniques are disclosed for a magnetic resonance elastography apparatus comprising a magnetic resonance unit and an elastography unit. The elastography unit has a vibration applicator, which has a vibration generator unit, and a coupling unit for coupling the elastography unit with the magnetic resonance unit. The elastography unit has a drive unit for generating a drive moment for the vibration generator unit, and the drive unit is embodied to be magnetic resonance-compatible.

### CROSS-REFERENCE TO RELATED APPLICATIONS

The present application claims the benefit of the filing date of European patent application no. EP19173755.0, filed on May 10, 2019, the contents of which are incorporated herein by reference in their entirety.

### TECHNICAL FIELD

The present disclosure relates to a magnetic resonance elastography apparatus with a magnetic resonance unit and an elastography unit.

### BACKGROUND

Tumor tissue and healthy tissue, in particular tissue which is free from tumors, have different vibration characteristics and/or a different excitation behavior during a vibration excitation. In elastography, diagnostic imaging uses the different vibration characteristics and/or the different excitation behavior between the different tissue types, in particular a healthy tissue and a tumor tissue. This different behavior can be represented in a magnetic resonance elastogram by means of magnetic resonance imaging.

### SUMMARY

The object underlying the present disclosure is in particular to provide a simple connection of an elastography unit to a magnetic resonance unit. The object is achieved by the various embodiments as described herein, as well as by means of the features of the claims.

The disclosure is based on a magnetic resonance elastography apparatus comprising a magnetic resonance unit and an elastography unit. The magnetic resonance unit has a main magnet, which is provided for generating a magnetic field, a gradient coil unit, which is provided for generating magnetic field gradients, and a radio-frequency coil unit, which is designed to radiate magnetic resonance sequences. The elastography unit has a vibration applicator, which has a vibration generator unit, and a coupling unit for coupling the elastography unit with the magnetic resonance unit. According to the disclosure, the elastography unit has a drive unit for generating a drive moment for the vibration generator unit, wherein the drive unit is embodied to be magnetic resonance-compatible.

The magnetic resonance elastography apparatus is designed to represent different vibration characteristics and/or a different excitation behavior of different tissue types, such as healthy, tumor-free tissue and tumor tissue, in a magnetic resonance elastogram. To this end, a region of the patient to be examined is excited by means of vibrations using the elastography unit. The different behavior can be represented in a magnetic resonance elastogram by means of magnetic resonance imaging.

To this end, the magnetic resonance elastography apparatus includes the magnetic resonance unit. The magnetic resonance unit includes a scanner unit and a patient receiving region surrounded e.g. cylindrically by the scanner unit. A patient or a region of the patient to be examined is disposed within the patient receiving region for a magnetic resonance elastography examination. The scanner unit includes a main magnet, the gradient coil unit, and the radio-frequency coil unit. A magnetic field generated by the main magnet is embodied in a homogeneous and constant manner, in particular within the patient receiving region and within a field of view (FOV) of the patient receiving region. The gradient coil unit is configured to generate magnetic field gradients during a magnetic resonance imaging process, wherein the magnetic field gradients are used for a spatial encoding during the magnetic resonance imaging process. The radio-frequency coil unit is provided to radiate radio-frequency magnetic resonance sequences into the patient receiving region, in particular into the FOV, of the magnetic resonance unit during a magnetic resonance scan.

The vibration applicator of the elastography unit is designed in particular to generate vibrations and/or pressure waves, and to transmit the generated vibrations and/or pressure waves to the patient and/or onto the patient. Here, the vibration applicator may be positioned in the vicinity of the patient, in particular in the vicinity of the region of the patient to be examined. Here, for example, the vibration applicator is held against the region of the patient to be examined During a magnetic resonance elastography examination, the vibration applicator of the elastography unit is therefore disposed within the patient receiving region, in particular within the FOV, and thus within the magnetic field of the magnetic resonance unit.

Furthermore, the elastography unit has a coupling unit for coupling the elastography unit with the magnetic resonance unit. The coupling unit may be provided to connect the elastography unit to the magnetic resonance unit. In particular, the coupling unit is designed to exchange control signals between the elastography unit and the magnetic resonance unit. Furthermore, the coupling unit can also be designed to connect the elastography unit electrically to the magnetic resonance unit, so that the elastography unit can be supplied with electrical energy via the coupling unit. By means of the coupling unit, an image data acquisition facility of the magnetic resonance unit can advantageously be coordinated with the elastography unit during a magnetic resonance elastography examination. In addition, the elastography unit can thus also be actuated by a control unit of the magnetic resonance unit and thus the magnetic resonance elastography examination can be controlled by means of a single control unit. Here, the coupling unit can comprise a cable-bound coupling unit. Moreover, a cableless and/or a wireless coupling unit is also possible.

A drive unit that is embodied to be magnetic resonance-compatible is to be understood to mean that the drive unit neither influences a magnetic resonance measurement nor interacts with the magnetic field and/or a gradient field and/or a radio-frequency field of the magnetic resonance unit. This embodiment of the disclosure makes it possible for the drive unit to be arranged in particularly close proximity to the vibration generator unit. This also makes it possible for a short transmission path to be provided for the transmission of a drive moment generated by the drive unit to the vibration generator unit and for long and error-prone transmission paths for transmitting the drive moment from the drive unit to the vibration generator unit to be dispensed with advantageously. Furthermore, a drive unit with a low power can also be made available in this way. This also enables a particularly cost-effective manufacture and/or design of the elastography unit.

A further advantage is that the elastography unit can thereby be constructed in a particularly compact and space-saving manner. This also enables the elastography unit to be operated and/or handled easily by medical operating personnel during a magnetic resonance elastography examination. In addition, a high level of patient comfort can be provided during a magnetic resonance elastography measurement on account of the compact design.

In an advantageous development of the disclosure, it can be provided that the drive unit comprises a piezo drive unit. Here, a piezo drive unit is to be understood in particular to mean a drive unit which uses the piezoelectric effect to produce and/or generate a drive moment. Here, a movement and/or a drive moment may be generated on the basis of a sliding or static friction between a fixed part (stator) and a moving part (rotor). Here, piezo drive units can also advantageously use a resonant vibration of the stator generated by piezoelectric solid-state actuators to generate the movement and/or the drive moment. It is particularly advantageous here that piezo drive units require no magnetic field to generate a movement and/or a drive moment, and thus there can be no interaction with and/or influence on the magnetic field of the magnetic resonance unit.

In an advantageous development of the disclosure, it can be provided that the drive unit is arranged within the vibration applicator. This can result in a direct transmission of the drive moment from the drive unit to the vibration generator unit. In addition, on the basis of this embodiment, it is advantageously possible to dispense with a long transmission unit, such as for example a transmission shaft for transmitting the drive moment from outside the patient receiving region to the vibration generator unit. In particular, it is thus advantageously possible to dispense with a rigid transmission unit between the drive unit and the vibration applicator. This also enables a particularly compact design of the elastography unit. In addition, it is thus possible to provide a particularly flexible deployment and/or a particularly flexible use of the vibration applicator for a magnetic resonance elastography examination.

In an advantageous development of the disclosure, it can be provided that the drive unit is arranged within the coupling unit. As a result, the vibration applicator of the elastography unit advantageously can have a particularly compact and simple design. It is thus possible to provide a high level of patient comfort and to advantageously reduce pressure on the patient during a magnetic resonance elastography examination due to the vibration applicator being held against the patient.

In an advantageous development of the disclosure, it can be provided that the elastography unit has a force transmission element, which transmits a drive moment generated by the drive unit to the vibration generator unit. This can advantageously result in a direct force transmission and/or a direct transmission of the drive moment from the drive unit to the vibration generator unit. If the drive unit is arranged within the vibration applicator, then preferably also the force transmission element is arranged within the vibration applicator so that the force transmission element can have a particularly compact and simple design. If, on the other hand, the drive unit is arranged outside the vibration applicator, such as for example within the coupling unit, then the force transmission element is preferably arranged between the drive unit and the vibration applicator. Advantageously, the force transmission element comprises a transmission shaft. In addition, the force transmission element, in an alternative embodiment of the disclosure, can also have further elements which appear expedient to the person skilled in the art, such as gear elements and/or toothed wheels, etc.

In an advantageous development of the disclosure, it can be provided that the force transmission unit has a flexible transmission shaft. In this connection, a flexible transmission shaft is to be understood to mean in particular a pliable and/or bendable transmission shaft, such as a flexible shaft. Such a transmission shaft can advantageously be deployed if a transmission path between two points, for example between the drive unit and the vibration generator unit, has a curved path and/or turns and does not have a straight path. In particular, in this way it is possible to provide a particularly compact elastography unit with fewer components, since it is possible to dispense with additional components such as additional force transmission elements and/or gear elements. In addition, in this way it is advantageously possible to achieve a higher level of patient comfort during a magnetic resonance elastography examination, since both a compact and lightweight vibration applicator and a compact transmission shaft with fewer components can be provided. Furthermore, the elastography unit can be manufactured particularly cost-effectively.

In an advantageous development of the disclosure, it can be provided that the coupling unit has a connector element, which is embodied to be compatible with a coil connector element of the magnetic resonance unit for receiving a coil connector of a local radio-frequency coil. Here, the connector element of the coupling unit may be configured to mate with, correspond to and/or to be of a similar type to a connector element of a local radio-frequency coil. In this way, the elastography unit can be coupled and/or connected to the magnetic resonance unit in a particularly simple manner. In this way, the elastography unit can in particular also be actuated directly by means of a control unit of the magnetic resonance unit, and thus the control unit of the magnetic resonance unit can advantageously be used to coordinate an elastography application being carried out by means of the elastography unit with a magnetic resonance imaging process being carried out by means of the magnetic resonance unit. In addition, it is advantageously possible for the elastography unit to be supplied with energy by means of the coupling unit, in particular the connector element of the coupling unit, of the elastography unit during a magnetic resonance elastography examination. The coil connector element of the magnetic resonance unit may be arranged on a patient positioning apparatus, on which the patient is positioned for a magnetic resonance imaging process. The patient positioning apparatus preferably comprises two or more coil connector elements, so that a local radio-frequency coil and the elastography unit can be used simultaneously during a magnetic resonance elastography examination. In addition, in this way a particularly secure and space-saving coupling and/or connection of the elastography unit to the magnetic resonance unit can be provided, as the coupling can take place directly on the patient positioning apparatus.

In an alternative embodiment of the disclosure, the coupling unit can also have a connector element embodied separately from a coil connector. To this end, the magnetic resonance unit and/or the patient positioning apparatus preferably also has a further connector element, which is embodied to complement the connector element of the coupling unit of the elastography unit.

In an advantageous development of the disclosure, it can be provided that the elastography unit has an electrical connection between the coupling unit and the vibration applicator. The electrical connection can comprise a wireless and/or a cableless connection. Particularly advantageously, however, the electrical connection between the coupling unit and the vibration applicator comprises a cable-bound electrical connection.

### BRIEF DESCRIPTION OF THE DRAWINGS/FIGURES

Further advantages, features and details of the disclosure will become apparent from the exemplary embodiments described below as well as with reference to the drawings, in which:

FIG. 1 shows a schematic diagram of an example magnetic resonance elastography apparatus with a magnetic resonance unit and an elastography unit, according to various aspects of the disclosure;

FIG. 2 shows a schematic diagram of an example of the elastography unit, according to various aspects of the disclosure; and

FIG. 3 shows a schematic diagram of an example elastography unit, according to various aspects of the disclosure.

### DETAILED DESCRIPTION

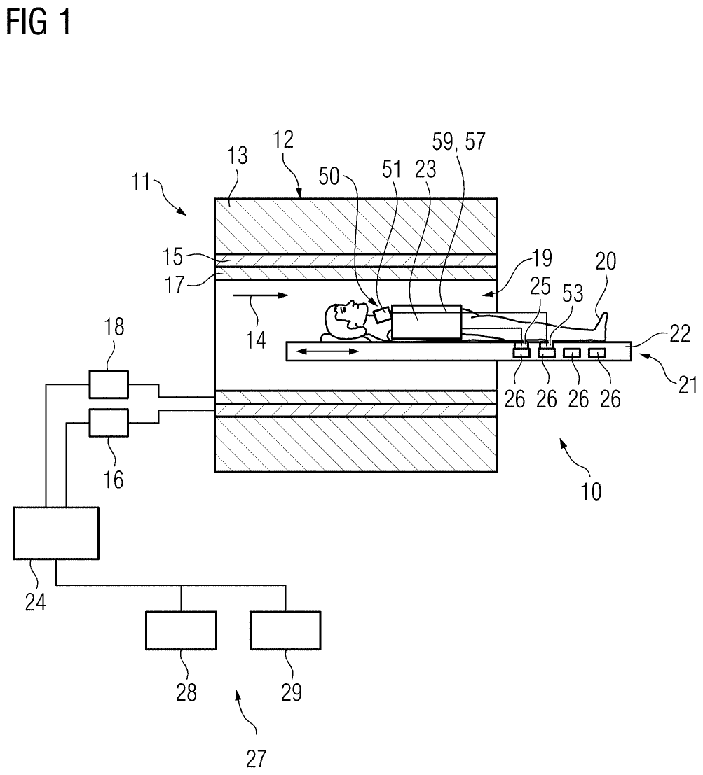

FIG. 1 shows a schematic diagram of an example magnetic resonance elastography apparatus with a magnetic resonance unit and an elastography unit, according to various aspects of the disclosure. The magnetic resonance elastography apparatus 10 comprises a magnetic resonance unit (or a magnetic resonance imager, device, or system) 11 and an elastography unit 50. The various units as shown and described herein may implement hardware (e.g., processors), software, or a combination of both. Thus, the various units as described herein may alternatively be referred to as “circuitry,” an “apparatus,” or a “device.”

The magnetic resonance unit (or MR imager) 11 comprises a scanner unit 12 formed by a magnet unit, which comprises a superconducting main magnet 13 for generating a strong and constant main magnetic field 14. The scanner unit 12 also has a gradient coil unit 15 for generating magnetic field gradients that are used for position encoding during an imaging process. The gradient coil unit is controlled by means of a gradient control unit 16 of the magnetic resonance unit 11. The scanner unit 12 further comprises a radio-frequency coil unit 17 for exciting a polarization, which is produced in the main magnetic field generated by the main magnet. The radio-frequency coil unit 17 is controlled by a radio-frequency coil control unit 18 of the magnetic resonance unit 11 and radiates radio-frequency magnetic resonance sequences into an examination space of the magnetic resonance unit 11, which examination space is substantially formed by a patient receiving region 19 of the magnetic resonance unit 11. Here, the radio-frequency coil unit 17 is integrated in a fixed manner within the scanner unit 12.

In addition, the magnetic resonance unit 11 has the patient receiving region 19 to accommodate a patient 20. In the present exemplary embodiment, the patient receiving region 19 is embodied cylindrically and is surrounded cylindrically in a peripheral direction by the scanner unit 12. In principle, however, it is conceivable for the patient receiving region 19 to be embodied differently. The patient 20 can be pushed and/or moved by means of a patient positioning apparatus 21 of the magnetic resonance unit 11 into the patient receiving region 19. For this purpose, the patient positioning apparatus 21 has a patient table 22, which is embodied such that it is able to move within the patient receiving region 19.

The magnetic resonance unit 11 further comprises at least one local radio-frequency coil 23, which is designed to receive magnetic resonance signals during a magnetic resonance examination. The local radio-frequency coil 23 is held against the body region of the patient 20 to be examined for a magnetic resonance examination. In the present exemplary embodiment, the local radio-frequency coil 23 is formed by a body coil. In principle, further embodiments of the local radio-frequency coil 23 in an alternative embodiment of the radio-frequency coils 23 are also conceivable, such as for example a head coil, a neck coil, a knee coil, etc.

For a transmission of the received magnetic resonance signals from the local radio-frequency coil 23 to the magnetic resonance unit 11, and in particular to a control unit 24 of the magnetic resonance unit 11, the local radio-frequency coil 23 has a connector element 25, in particular a coil connector. The connector element 25, in particular the coil connector of the local radio-frequency coil 23 is configured to be compatible with a coil connector element 26 of the magnetic resonance unit 11. The coil connector element 26 may be configured to receive the connector element 25, in particular the coil connector, of the local radio-frequency coil 23. The coil connector element 26 may be arranged on the patient positioning apparatus 21 and connected via the patient positioning apparatus 21 to the control unit 24 of the magnetic resonance unit 11. The patient positioning apparatus 21 may comprise two or more coil connector elements 26 for connecting and/or coupling two or more local radio-frequency coils simultaneously during a magnetic resonance examination. From the patient positioning apparatus 21, the magnetic resonance signals are transmitted to the control unit 24 by means of a data transmission unit (not shown in detail) of the magnetic resonance unit 11.

For controlling the main magnet 13, the gradient control unit 16, and for controlling the radio-frequency coil control unit 18, the magnetic resonance unit 11 includes the control unit 24. The control unit 24 may include one or more processors, hardware, and/or software, and may be configured to centrally control the magnetic resonance unit 11, such as for example the performance of a predetermined imaging gradient echo sequence. Furthermore, the control unit 24 comprises an evaluation unit (not shown in detail) for evaluating medical image data, which is acquired during the magnetic resonance examination.

Furthermore, the magnetic resonance unit 11 comprises a user interface 27, which is connected to the control unit 24. Control information, such as for example imaging parameters and reconstructed magnetic resonance images, can be displayed on a display unit 28, for example on at least one monitor, of the user interface 27 for medical operating personnel. In addition, the user interface 27 has an input unit 29 by means of which information and/or parameters can be input by the medical operating personnel during a scanning procedure.

The magnetic resonance unit 11 as shown can naturally comprise further, fewer, or alternate components that magnetic resonance units 11 typically have. A general mode of operation of a magnetic resonance unit 11 is moreover known to the person skilled in the art, so that a detailed description of the further components is dispensed.

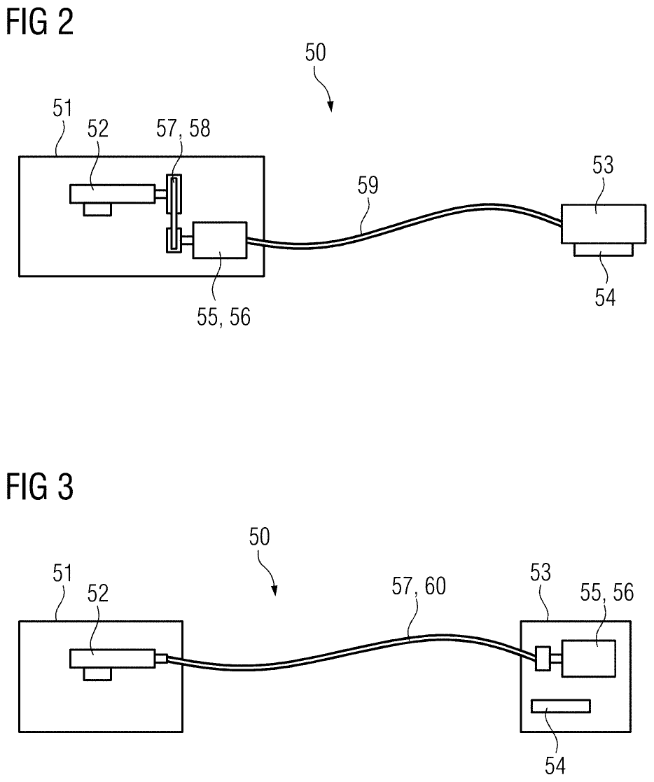

The elastography unit 50 of the magnetic resonance elastography apparatus 10 comprises a vibration applicator 51, which has a vibration generator unit 52 (FIGS. 2 and 3). During a magnetic resonance elastography examination of a patient 20, the vibration applicator 51 is arranged directly on the patient 20, in particular on the region of the patient 20 to be examined.

Furthermore, the elastography unit 50 has a coupling unit 53 (e.g., a coupler) for coupling and/or connecting the elastography unit 50 with the magnetic resonance unit 11. The coupling unit 53 has a connector element 54, which in the present exemplary embodiment is embodied to be compatible with one of the coil connector elements of the magnetic resonance unit 11, in particular of the patient positioning apparatus 21 of the magnetic resonance unit 11 (FIGS. 2 and 3). The elastography unit 50 is thus actuated by means of the control unit 24 of the magnetic resonance unit 11, wherein the control signals from the magnetic resonance unit 11, in particular from the control unit 24, are transmitted via the coupling unit 53 to the elastography unit 50. Furthermore, energy is supplied to the elastography unit 50 by means of the coupling unit 53, in particular the connector element 54 of the elastography unit 50 in a connected state of the coupling unit 53, in particular of the connector element 54.

Moreover, the elastography unit 50 includes a drive unit (e.g. a driver) 55. The drive unit 55 is designed to generate a drive moment for the vibration generator unit 52 during an elastography examination. Here, the drive unit 55 is embodied to be magnetic resonance-compatible. Here, the drive unit 55 is embodied such that the drive unit 55 neither influences a magnetic resonance measurement nor interacts with the magnetic field and/or a gradient field and/or a radio-frequency field of the magnetic resonance unit 11. The magnetic resonance-compatible drive unit 55 may comprise for instance a piezo drive unit 56, in which the piezoelectric effect is used to generate a drive moment (FIGS. 2 and 3).

FIG. 2 shows a first exemplary embodiment of the elastography unit 50 in detail. In this exemplary embodiment, the drive unit 55 described with reference to FIG. 1. The piezo drive unit 56 is arranged within the vibration applicator 51. Because the piezo drive unit 56 is embodied to be magnetic resonance-compatible, there is no possibility of it influencing and/or interacting with the magnetic field and/or the gradient field and/or the radio-frequency field of the magnetic resonance unit 11.

The elastography unit 50 in FIG. 2 also has a force transmission element 57, which transmits a driving force and/or a drive moment from the drive unit 55, e.g. the piezo drive unit 56, to the vibration generator unit 52. On account of the arrangement of the drive unit 55, in particular of the piezo drive unit 56 within the vibration applicator 51, the force transmission element 57 is also arranged within the vibration applicator 51. Here, the force transmission element 57 comprises a transmission shaft 58. On account of the arrangement of the drive unit 55, in particular of the piezo drive unit 56 within the vibration applicator 51, a particularly compact force transmission element 57, in particular a compact transmission shaft 58, can be used. Moreover, in a further development of the disclosure, the force transmission element 57 can also have an alternative embodiment to a transmission shaft 58, such as for example a toothed wheel and/or a gear element, etc.

The elastography unit 50 further has an electrical connection 59, which is arranged between the coupling unit 53 and the vibration applicator 51, in order to transmit control signals from the control unit 24 of the magnetic resonance unit 11 to the vibration applicator 51 and/or to enable the vibration applicator 51 to be supplied with energy via the magnetic resonance unit 11.

The coupling unit 53 of the elastography unit 50 shown in FIG. 2 is implemented in accordance with the explanations for the coupling unit 53 as shown and described with respect to FIG. 1. In addition, an embodiment of the vibration generator unit 52 is also embodied in accordance with the explanations for FIG. 1.

FIG. 3 shows an alternative exemplary embodiment of the elastography unit 50. In principle, components, features and functions remaining substantially the same are identified with the same reference characters. The following description includes the differences from the exemplary embodiment in FIG. 2, with reference being made to the description of the exemplary embodiment in FIG. 2 with respect to components, features, and functions that remain the same.

The elastography unit 50 shown in FIG. 3 has a drive unit 55, in particular a piezo drive unit 56, which is arranged within the coupling unit 53. As a result, the vibration applicator 51 has a particularly compact design and thus enables a high level of patient comfort during a magnetic resonance elastography examination.

In addition, the elastography unit 50 in FIG. 3 includes a force transmission element 57. In the present exemplary embodiment, the force transmission element 57 transmits a driving force and/or a drive moment from the drive unit 55, in particular the piezo drive unit 56, within the coupling unit 53 to the vibration generator unit 52 within the vibration applicator 51. The force transmission element 57 is thus arranged between the coupling unit 53 and the vibration applicator 51. The force transmission element 57 has a transmission shaft 60, wherein in the present exemplary embodiment the force transmission element 57, in particular the transmission shaft 60, has a flexible transmission shaft 60, such as for instance a flexible shaft. The flexible transmission shaft 60 is embodied in particular to be bendable and/or pliable, so that the vibration applicator 51 can be positioned on the patient 20 in a flexible manner in relation to the coupling unit 53.

The connector element 54 of the coupling unit 53 of the elastography unit 50 shown in FIG. 2 may be implemented in accordance with the explanations for the coupling unit 53 as shown and described herein with respect to FIG. 1. In addition, an embodiment of the vibration generator unit 52 may also be implemented in accordance with the explanations for FIG. 1.

The elastography units 50 shown in FIGS. 1 to 3 can naturally comprise further, fewer, or alternate components in which elastography units 50 typically have. A general mode of operation of an elastography unit 50 is moreover known to the person skilled in the art, so that a detailed description of the further components is dispensed.

Although the disclosure has been illustrated and described in detail on the basis of the preferred exemplary embodiments, the disclosure is not limited by the disclosed examples, and other variations may be derived by the person skilled in the art without leaving the scope of protection of the disclosure.

What is claimed is:

1. A magnetic resonance (MR) elastography apparatus, comprising:
a magnetic resonance imager including a main magnet, gradient coil circuitry, and radio-frequency (RF) coil circuitry; and
an elastography device including a vibration applicator, the vibration applicator including a vibration generator and a coupler configured to couple the elastography device to the magnetic resonance imager,
wherein the elastography device includes a driver configured to generate a drive moment for the vibration generator, and
wherein the driver is magnetic resonance compatible.

2. The MR elastography apparatus as claimed in claim 1, wherein the driver is magnetic resonance compatible by neither (i) influencing a magnetic resonance measurement performed by the by the magnetic resonance imager, nor (ii) interacting with a magnetic field, a gradient field, or a radio-frequency field generated by the magnetic resonance imager.

3. The MR elastography apparatus as claimed in claim 1,
wherein the driver comprises a piezo driver.

4. The MR elastography apparatus as claimed in claim 1,
wherein the driver is arranged within the vibration applicator.

5. The MR elastography apparatus as claimed in claim 1,
wherein the driver is arranged within the coupler.

6. The MR elastography apparatus as claimed in claim 1,
wherein the elastography apparatus includes a force transmission element configured to transmit a drive moment generated by the driver to the vibration generator.

7. The MR elastography apparatus as claimed in claim 6,
wherein the drive transmission element comprises a transmission shaft.

8. The MR elastography apparatus as claimed in claim 6,
wherein the drive transmission element includes a flexible transmission shaft.

9. The MR elastography apparatus as claimed in claim 1,
wherein the coupler includes a connector element that is compatible with a coil connector element of the magnetic resonance imager for receiving a coil connector of a local RF coil associated with the magnetic resonance imager.

10. The MR elastography apparatus as claimed in claim 1,
wherein the elastography apparatus includes an electrical connection between the coupler and the vibration applicator.
